(19) United States
(12) Patent Application Publication
(10) Pub. No.: US 2024/0390602 A1
(43) Pub. Date: Nov. 28, 2024

(54) Component for a drug delivery device and drug delivery device

(71) Applicant: Sanofi, PARIS (FR)

(72) Inventors: Paul Richard Draper, Warwick, Warwickshire (GB); Anthony Paul Morris, Warwick, Warwickshire (GB); Samuel Keir Steel, Warwick, Warwickshire (GB)

(21) Appl. No.: 18/694,116

(22) PCT Filed: Sep. 22, 2022
(86) PCT No.: PCT/EP2022/076300

§ 371 Date: Mar. 21, 2024

(30) Foreign Application Priority Data
Sep. 24, 2021 (EP) 2135183.0

(51) Int. Cl. A61M 5/315

(52) U.S. Cl. CPC A61M 5/31576

(57) ABSTRACT

The disclosure relates to a component for a drug delivery device which is configured to be connected to an arrangement of the drug delivery device and which includes a functional element. At least when the component is connected to the arrangement, a transition region is formed between facing surfaces of elements of the drug delivery device, wherein at least one of these elements is an element of the component. The transition region reaches from the exterior of the component to the interior of the component and is designed such that the risk of fluid reaching from the exterior into the interior of the component via the transition region is reduced in order to protect the functional element.

### CROSS REFERENCE TO RELATED APPLICATIONS

The present application is the national stage entry of International Patent Application No. PCT/EP2022/076300, filed on Sep. 22, 2022, and claims priority to Application No. EP 21315183.0, filed on Sep. 24, 2021, the disclosures of which are incorporated herein by reference.

### TECHNICAL FIELD

A component for a drug delivery device is provided. Furthermore, a drug delivery device is provided.

### BACKGROUND

Administering an injection is a process which presents a number of risks and challenges for users and healthcare professionals, both mental and physical. A drug delivery device may aim to make self-injection easier for patients. Drug delivery devices using electronics are becoming increasingly popular in the pharmaceutical industry as well as for users or patients. A stable operation of the electronic components is desirable in order to guarantee that the drug delivery device operates correctly.

### SUMMARY

In one aspect, the present disclosure describes an improved component for a drug delivery device. For example, the component may allow a stable operation of the drug delivery device. In another aspect, the present disclosure describes an improved drug delivery device.

Firstly, the component for a drug delivery device is specified. The component may be a user interface member, like a button and/or a knob, for operating and/or activating the drug delivery device by a user. The component may also be module for a drug delivery device which is not operated by a user.

According to at least one embodiment, the component is configured to be connected to an arrangement of the drug delivery device. For example, the component is configured to be permanently connected to the arrangement or to be releasably connected to the arrangement.

For example, the component is configured to be arranged at a proximal end of the arrangement. The component may form the proximal end of the drug delivery device when connected to the arrangement.

The arrangement may include a drug container holder for holding a drug container filled with a drug. The arrangement may include a drive mechanism for delivering the drug and/or a dose-setting mechanism for dialing a drug dose. The mechanism(s) may include drive elements, like a plunger rod and/or a drive sleeve and/or a dial sleeve. The drive elements are, e.g., operatively coupled in order to move the plunger rod and/or the drive sleeve and/or the dial sleeve axially and/or rotationally in order to deliver the drug and/or in order to dial the dose of the drug.

The component may be connectable to the arrangement, e.g., such that the component is operatively coupled to the drive mechanism and/or dose dial or setting mechanism. When coupled to the arrangement, the component may be movable, e.g., rotatable and/or axially movable with respect to at least one element, e.g., the drug container holder, of the arrangement.

According to at least one embodiment, the component includes at least one functional element. The functional element may, e.g., be associated with an electric or electronic functionality of the component and/or a mechanical functionality of the component. The functional element may be arranged in an interior of the component. For example, at least when connected to the arrangement, the functional element may be hidden behind one or more housing elements of the component and/or the arrangement. For example, the functional element may then not be freely accessible and/or visible from outside of the drug delivery device.

According to at least one embodiment, at least when the component is connected to the arrangement, a transition region is formed between facing surfaces of elements of the drug delivery device. At least one of these elements may be an element of the component, e.g., a housing element of the component.

The transition region may be formed between two or more elements, e.g., between three or more elements of the drug delivery device. All of these elements may be elements of the component so that the component itself includes the transition region or one or more of the elements defining the transition region may be elements of the arrangement. The elements between which the transition region is formed may each be formed in one piece and/or may be formed of plastic. In some embodiments, the elements are separate elements, i.e. they are not formed in one common piece.

The transition region is formed between surfaces which face each other. The surfaces may, at least sectionally, run parallel to each other. The surfaces may be spaced from each other so that a gap is formed between the elements. Alternatively, the surfaces may contact each other so that an interface is formed between the elements.

According to at least one embodiment, the transition region reaches from the exterior of the component to the interior of the component, e.g., from an outer surface of the component to an inner surface of the component, wherein the inner surface adjoins the interior. For example, the gap and/or the interface may reach from the exterior to the interior. For example, the transition region is accessible and/or visible from the exterior of the component. The interior of the component is, e.g., circumferentially surrounded by at least one housing element of the component. This means, the interior may be delimited by at least one housing element of the component in an outward radial direction.

In this context, the “exterior of the component” may be a space or area outside of the housing element of the component. The “exterior of the component” may be a space or area inside the housing of the drug delivery device. Alternatively or additionally, the “exterior of the component” may be a space or area outside the housing of the drug delivery device.

In this context, the “outer surface of the component” may be a surface of the component, whose surface normal does not pass through another surface of the component.

According to at least one embodiment, the transition region is designed such that the risk of fluid reaching from the exterior into the interior of the component via the transition region is reduced in order to protect the functional element. The fluid may be in liquid form or in gas form. The fluid may be water or the drug of the drug delivery device.

The length of the transition region, measured from the exterior to the interior of the component, may be at least 1 mm, at least 1.5 mm, or at least 2 mm.

There may be two or more transition regions, e.g., in the component or at least when the component is connected to the arrangement. All features disclosed in connection with one transition region are also disclosed for the other transition regions.

In at least one embodiment, the component for a drug delivery device is configured to be connected to an arrangement of the drug delivery device and includes a functional element. At least when the component is connected to the arrangement, a transition region is formed between facing surfaces of elements of the drug delivery device, wherein at least one of these elements is an element of the component. The transition region reaches from the exterior of the component to the interior of the component and is designed such that the risk of fluid reaching from the exterior into the interior of the component via the transition region is reduced in order to protect the functional element.

Fluid reaching functional elements, e.g., elements which are associated with electric or electronic functionalities of the component, may affect the functionality of the elements. For a component configured to be connected to an arrangement, e.g., when this component is arranged to be movable with respect to at least one element the arrangement, results in a transition region between one or more elements of the component and one or more elements of the arrangement which reaches from the exterior to the interior of the component. Such a transition region, however, is a region through which fluid could potentially enter from the exterior into the interior of the component.

Moreover, transition regions may be formed between elements of the component. For example, during manufacturing of the component, it may be necessary to physically connect to the functional element, e.g., to program, calibrate, configure, and/or check its functionality. Such a functional element could be located behind a cover element of the component which is added at the end of the manufacturing. However, a transition region then forms between this cover element and a further housing element of the component through which again fluid could potentially enter into the interior of the component.

Damage of functional elements of the components can, inter alia, be reduced by designing the transition region between elements such that the risk of fluid reaching from the exterior to the interior via the transition region is reduced. Different ways for reducing this risk are possible and are further described in the following.

The component and/or the drug delivery device specified herein may be elongated and/or may include a longitudinal axis, e.g., a main extension axis. Additionally or alternatively, the component and/or the drug delivery device may have a rotational symmetry with respect to the longitudinal axis. A direction parallel to the longitudinal axis is herein called an axial direction. By way of example, the drug delivery device and/or the component may be cylindrically-shaped.

Furthermore, the drug delivery device may include an end, e.g., a longitudinal end, which may be provided to face or to be pressed against a skin region of a human body. This end is herein called the distal end. A drug or medicament may be supplied via the distal end. The opposing end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device, e.g., of the component, is herein understood to be the end of the member/element/feature located most distally. Accordingly, the proximal end of a member or element or feature is herein understood to be the end of the element/member/feature located most proximally.

In other words, distally is used herein to specify directions, ends or surfaces which are arranged or are to be arranged to face or point towards a dispensing end of the drug delivery device or components thereof and/or point away from, are to be arranged to face away from or face away from the proximal end. On the other hand, proximal is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end and a distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis. The term “angular direction”, “azimuthal direction” or “rotational direction” are herein used as synonyms. Such a direction is a direction perpendicular to the longitudinal axis and perpendicular to the radial direction.

According to at least one embodiment, the transition region is a fluid path transition region in which the facing surfaces are spaced from each other so that a fluid path extends between the surfaces. Fluid can travel along this fluid path. The fluid path may, e.g., reach from the exterior of the component into the interior of the component, e.g., up to the functional element.

In some embodiments, the transition region includes a gap or space between the elements defining the transition region. The gap may extend without interruption from the exterior of the component to the interior of the component. A fluid path can extend in this gap. For example, the minimal height or width of the gap, measured between the facing surfaces of the elements, is at least 0.05 mm, 0.1 mm or 0.2 mm. Additionally or alternatively, the maximal height or width of the gap is at most 1.5 mm, 1 mm, 0.5 mm, 0.4 mm, or 0.3 mm.

According to at least one embodiment, the transition region is designed such that, inside the transition region, the fluid path includes two sections which extend in different axial directions. This means that fluid, e.g. water or the drug in the device, which flows inside the transition region along the fluid path from the exterior to the interior has to change its flow direction from one axial direction to the opposite axial direction. For example, when following the fluid path inside the transition region from the exterior to the interior, a section extending in proximal direction follows a section extending distal direction. The sections extending in different axial directions may run, at least sectionally, parallel to each other. The two sections of the fluid path may be radially offset to each other. For example, the section extending in proximal direction may be radially inwardly offset with respect to the section running distal direction.

For example, the two sections each have a length along an axial direction of at least 2 mm or at least 5 mm. Additionally or alternatively, the lengths of the sections along an axial direction may be at most 1 cm or at most 5 mm.

According to at least one embodiment, the length of the fluid path is at least 1 mm or at least 2 mm or at least 7 mm or at least 8 mm or at least 9 mm or at least 10 mm. Alternatively or additionally, the fluid path may have a length which is at most 20 mm or at most 18 mm or at most 15 mm or at most 13 mm or at most 5 mm or at most 3 mm. The length of the fluid path may be measured in a position when the component is in an initial position relative to a housing or body of the drug delivery device, e.g. before a dose setting operation is commenced and/or after a dose delivery operation has been completed. During dose setting, the length may be reduced.

According to at least one embodiment, inside the transition region, the fluid path includes at least one coil or meander, respectively, and/or is a tortuous path. For example, inside the transition region, the fluid path includes at least one curved section, e.g., between the two sections extending in different axial directions. The fluid path inside the transition region may include two or more coils or meanders. Hence, the fluid path can be formed as a labyrinth-type path inside the transition region.

Forming a transition region such that a gap or space is formed between the facing surfaces of the two elements may be advantageous, e.g., when a relative movement between the elements defining the transition region is desired. Designing the transition region such that a fluid path through which fluid can potentially reach into the interior of the component has at least two sections running in different axial directions and/or having at least one coil inside the transition region reduces the probability that indeed fluid travels the whole distance along the fluid path.

According to at least one embodiment, the component is configured to be axially and/or rotationally moved relative to at least one element of the arrangement during usage of the drug delivery device. Thus, when the component is connected to the arrangement, the component is still movable with respect to at least one element of the arrangement. For example, a dose dial and/or a drug delivery process is associated with such a movement.

According to at least one embodiment, the length of the fluid path inside the transition region changes during movement of the component, e.g., by at least 5% or at least 10%. For example, the transition region is formed between at least one element of the component and at least one element of the arrangement, wherein the element of the component is movable relative to the element of the arrangement. Thus, the surfaces of the elements between which the transition region is formed are moved with respect to each other during movement of the components.

For example, during movement of the component, one section of the fluid path inside the transition region which extends in an axial direction changes its length, e.g., doubles its length or halves its length. The length of the section inside the transition region extending in the different axial direction may stay constant during the movement or may also change, e.g., double or halves its length.

According to at least one embodiment, the transition region is formed between elements of both, the component and the arrangement. A transition region in the form of a fluid path transition region may be formed between elements of the arrangement and the component, e.g., if these elements are movable with respect to each other when the component is connected to the arrangement.

At least one element of the component and at least one element of the arrangement defining the transition region or a portion thereof may be housing elements, i.e., elements delimiting the component or the arrangement, respectively, in a direction, e.g., in outward radial direction and/or in an axial direction. For example, the element of the arrangement is a housing element. The element of the component may be a grip element. For example, the element of the arrangement and the element of the component between which a least a portion of the transition region is formed may each form an outer surface of the drug delivery device touchable by a user.

According to at least one embodiment, the transition region is formed between a distal section of an element of the component and a proximal section of an element of the arrangement. The distal section may extend from the rest of the element in the distal direction and/or may define a distal end of the element. Accordingly, the proximal section may extend in the proximal direction from the rest of the element and/or define a proximal end of the element.

According to at least one embodiment, when the component is connected to the arrangement, the distal section of the element of the component and the proximal section of the element of the arrangement axially overlap and/or are radially offset from each other. The distal section and the proximal section may also overlap in angular direction. The distal section of the element of the component may, for example, be offset in the radial inward direction with respect to the proximal section of the element of the arrangement.

The functional element(s) may be offset in the proximal direction from the distal section of the element.

According to at least one embodiment, the transition region is formed between at least three elements. A first section of the transition region may be formed between a first element and a second element and a second section of the transition region may be formed between the second element and the third element. For example, the second element is arranged radially between the first and the third element. The second element may axially and/or rotationally overlap with the first and/or the third element. By way of example, the arrangement includes the first and the third element, and the component includes the second element. The second element may be the element including the distal section. The first element may be the element including the proximal section. The first and the second element may be housing elements. The fluid path transition region with a fluid path having two sections extending in opposite axial directions may extend between the three elements.

According to at least one embodiment, the transition region is at tight transition region in which the facing surfaces are tightly fitted to each other so that no fluid can pass through the transition region. For example, in the transition region, the surfaces touch each other (i.e., the distance is zero) and/or a distance between the surfaces is at most 0.5 mm or at most 0.1 mm or at most 0.05 mm in order to make the transition region tight. Such a tight transition region can be achieved by an interference fit between the elements providing the surfaces which face one another. For example, pressure is generated, e.g. between the two elements having the surfaces which are fitted together by the interference fit. Hence, the transition region can withstand regular fluid pressures without opening up into a bigger gap. Additionally or alternatively, a sealing material may be located inside the transition region to make it tight.

With such a tight transition region, it can be prevented that fluid reaches from the exterior into the interior via this transition region.

According to at least one embodiment, the tight transition region has a length, measured between the exterior and the interior of the component, of at least 1 mm or at least 1.5 mm or at least 2 mm. For example, the tight transition region is tight over its entire length or over at least 20% or at least 50% of its length.

The tight transition region may include a tight subregion and a non-tight subregion. In the non-tight subregion, the gap between the facing surfaces may be larger than in the tight subregion, e.g., large enough that a fluid can be arranged in the non-tight subregion. A tight subregion may be located closer to the exterior than the non-tight subregion, e.g., may adjoin the exterior.

According to at least one embodiment, a snap connection between the two elements defining the transition region is formed in the transition region. For example, the snap connection is formed at an end of the transition region, e.g., at the end closer to the interior of the component. The snap connection may mechanically fix the two elements to each other, e.g., such that they are not movable with respect to each other. The snap connection may be formed by a protrusion of one element engaging into a recess of another element.

According to at least one embodiment, the transition region is formed between elements of the component. For example, these elements of the component are not movable with respect to each other. For example, in this case, a transition region in the form of a tight transition region may be realized. The elements of the component defining the transition region or at least a portion thereof may be housing elements. For example, one of these elements may be a grip element forming a lateral surface of the component and one of the elements may be a cover element forming a proximal surface of the component.

According to at least one embodiment, the functional element is axially and/or radially offset from the transition region, e.g., from all transition regions. When the functional element is axially offset from the transition region, there may be no overlap between the functional element and the transition region in axial direction. Similarly, when the functional element is radially offset from the transition region, there may be no overlap between the functional element and the transition region in radial direction. Similarly, when the functional element is axially and radially offset from the transition region, there may be no overlap between the functional element and the transition region in axial and radial direction. In this context “no overlap” may mean that no part or portion of the functional element overlaps with no part or portion of the transition region in the respective direction(s).

According to at least one embodiment, at least when the component is connected to the arrangement, at least two transition regions are formed. For example, one transition region is formed by elements of both, the arrangement and the component. A further transition region may be formed between elements of the component.

According to at least one embodiment, at least one transition region is a fluid path transition region as specified above and at least one transition region is a tight transition region as specified above. For example, the fluid path transition region is arranged distally with respect to the tight transition region. The tight transition region may be formed at a proximal end of the component. The fluid path transition region may be formed at a distal end of the component.

According to at least one embodiment, the functional element(s) is (are) axially arranged between the fluid path transition region and the tight transition region. For example, the functional element(s) does (do) neither axially overlap with the fluid path transition region nor with the tight transition region.

According to at least one embodiment, a surface of the component in the vicinity of the transition region is formed of a hydrophobic material. For example, a surface of the component which defines a transition region, i.e., which adjoins the transition region, is formed of a hydrophobic material. The component may include a coating of a hydrophobic material forming this surface.

Using a hydrophobic surface in or in the vicinity of the transition region may also reduce the risk of fluid reaching into the interior via the transition region. “In the vicinity” means, e.g., that the hydrophobic surface is at a distance of at most 1 mm or at most 0.5 mm from the transition region. The hydrophobic surface may also be an outer surface of the component and/or the arrangement.

According to at least one embodiment, the functional element is associated with an electric or electronic functionality of the component. For example, the component may include one or more electric or electronic elements and may have an electric or electronic functionality. The at least one functional element may be an electric element. For example, the component may be configured to measure a drug dose delivered during usage of the drug delivery device. The component may furthermore be configured to communicate the measured dose to an external device, like a smart phone or a computer, e.g., via a Bluetooth connection. For this purpose, the component may include a wireless communication interface, i.e. a Bluetooth interface. Additionally or alternatively, the component may include electric or electronic user communication elements, like one or more LEDs, to audibly and/or visually communicate a state of the drug delivery device to the user.

According to at least one embodiment, the functional element is a sensor, e.g. an optical sensor, or an electro-mechanical switch or a circuit board or a battery or a LED. The component may include one or more or all of the mentioned functional elements. The optical sensor may include an LED, e.g. an infrared LED, and a sensor element configured to detect a reflected portion of the light emitted by the LED. The sensor may, e.g., be configured to detect a relative movement, especially a relative rotation, between the sensor and a further element, e.g. an element of the arrangement. For example, the sensor is a sensor for reading a Gray-Code.

According to at least one embodiment, the component is a user interface member configured to be touched by a user in order to operate the user interface member when the user interface member is connected to the arrangement. For example, the component may be a knob or a button, permanently connected or connectable to the arrangement or releasably connectable to the arrangement. The user interface member may be configured to perform a dose dial and/or a drug delivery when connected to the arrangement and when operated by a user.

For example, the user interface member includes a lateral surface which forms an outer surface of the component and which is configured to be grabbed by a user. The lateral surface may delimit the user interface member in outward radial direction. For example, the lateral surface may run parallel or acute-angled to the longitudinal axis.

The lateral surface may be configured to be grabbed by a user using two fingers for performing a rotation of the user interface member around the longitudinal axis, e.g., with respect to the housing element of the arrangement. Additionally or alternatively, the user interface member may include a proximal surface facing in proximal direction. The proximal surface may run perpendicularly or obliquely with respect to the longitudinal axis and/or the lateral surface. The proximal surface may be configured to be touched by a user, e.g., using only one finger, e.g., for pushing the user interface member in distal direction.

According to at least one embodiment, the lateral surface may include gripping features, e.g. grooves. The grooves may extend parallel or acute-angled to the longitudinal axis. The gripping features may simplify grabbing of the user interface member by a user.

Next, the drug delivery device is specified. The drug delivery device may be an injection device and/or a pen type device, e.g. a dial extension pen. The drug delivery device may be a variable dose device in which the drug dose to be delivered to a user can be variably set. For example, the drug delivery device is a reusable device. The drug delivery device may be configured to perform several drug delivery processes one after the other. For example, during each such drug delivery process, a dialled or set dose is delivered to a user.

According to at least one embodiment, the drug delivery device includes a component as specified above. Therefore, all features disclosed for the component are also disclosed for the drug delivery device and vice versa.

According to at least one embodiment, the drug delivery device includes an arrangement with a container holder for holding a drug container. The container holder may be a housing element of the arrangement or may be connected or connectable to the housing element. The container holder may be configured to hold the drug container axially and/or rotationally fixed with respect to the housing element of the arrangement. For example, the container holder may hold the drug container such that the drug container does not move in an axial and/or a rotational direction during a drug delivery process.

According to at least one embodiment, the component is a user interface member configured to be operated by the user in order to perform a dose dial and/or a dose delivery process.

According to at least one embodiment, the component is configured to be rotated and/or axially moved relative to the container holder when operated by a user.

According to at least one embodiment, the drug delivery device includes a drug container filled with a drug. The drug container may be a syringe with a pre-mounted needle at a distal end. Alternatively, a needle may be attachable to the drug container, e.g. a distal end thereof.

A method for operating the drug delivery device may be as follows: The component in form of a user interface member, e.g. a knob, and connected to the arrangement is grabbed by a user, e.g. at the lateral surface, and rotated thereby dialing or setting a dose to be injected to the user. The user interface member may be rotated on a helical path with respect to the housing and/or the drug container (holder), thereby moving e.g. in the proximal direction. After having dialed the desired dose, the user interface member may be pushed in the axial direction, e.g. in the distal direction, and the dose of the drug is injected. For this purpose, a user may press against the proximal surface of the user interface member. During movement of the user interface member in the distal direction, the user interface member may not rotate but a movable element of the arrangement, e.g. an element of a dose setting and/or drive mechanism of the device, may rotate. The dialed dose may thereby be ejected, e.g. injected into a patient. The sensor(s) in the user interface member may measure the rotation of the movable element. The measurement signals of the sensor(s) may then be sent to an electric element, e.g. a processor, of the user interface member which may determine from the measurement signal(s) the delivered dose. This information may then be communicated to a further device, e.g. with help of the wireless communication module.

Features disclosed in conjunction with the method do also apply for the component and the device and vice versa.

Hereinafter, the component and the drug delivery device described herein will be explained in more detail with reference to drawings on the basis of exemplary embodiments. Same reference signs indicate same elements in the individual figures. However, the size ratios involved are not necessarily to scale, individual elements may rather be illustrated with exaggerated size for better understanding.

### BRIEF DESCRIPTION OF THE DRAWINGS

FIG. 1 shows an exemplary embodiment of a drug delivery device in an exploded view,

FIGS. 2 and 3 show proximal sections of an exemplary embodiment of the drug delivery device in different views.

FIG. 4 illustrates a dose set situation of the device.

FIG. 5 illustrates additional fluid paths in the device of FIGS. 1 to 4.

### DETAILED DESCRIPTION

In the following, exemplary embodiments will be described with reference to an insulin injection device. The present disclosure is however not limited to such application and may equally well be deployed with injection devices that are configured to eject other medicaments or with drug delivery devices in general, e.g., pen-type devices and/or injection devices.

Moreover, in the following, exemplary embodiments will be described, in which the component is a user interface member configured to be operated by a user for dose setting and/or dose injection. However, the present disclosure is not limited to a component in form of a user interface member. Rather, the component may also be a module which is connected or connectable to an arrangement of the drug delivery device, e.g. for measuring a dialed dose, but which is not foreseen to be operated or moved by a user.

Certain exemplary embodiments in this document are illustrated with respect to a drug delivery device in form of an injection device where the component in form of the user interface member is a knob which realizes an injection button and a dose setting (dialing) member at the same time, e.g. similar to the devices disclosed in WO 2014/033195 A1 or WO 2014/033197 A1 (the disclosure content of these documents is explicitly incorporated into the present document by reference, e.g., as far as electronic functionalities and the operation of the dose setting and/or drive mechanism is concerned). Thus, the knob may be used for initiating and/or performing a dose delivery operation of the drug delivery device and may also be used for initiating and/or performing a dose setting operation. The devices may be of the dial extension type, i.e. their length increases during dose setting. Other injection devices with the same kinematical behaviour of the dial extension during dose setting and dose expelling operational mode are known as, for example, the Kwikpen® or Savvio® device marketed by Eli Lilly and the FlexPen®, FlexTouch® or Novopen® device marketed by Novo Nordisk. An application of the general principles to these devices therefore appears straightforward and further explanations will be omitted. However, the general principles of the present disclosure are not limited to that kinematical behaviour.

Certain other embodiments may be conceived for application to injection devices where there are separate injection button and grip components/dose setting members the device disclosed in WO 2004/078239 A1. Thus, the present disclosure also relates to systems with two separate user interface members, e.g., one for the dose setting operation and one for the dose delivery operation. In order to switch between a dose setting configuration of the device and a dose delivery configuration, the user interface member for dose delivery may be moved relative to the user interface member for dose setting.

If one user interface member is provided, the user interface member may be moved distally relative to a housing element. In the course of the respective movement, a clutch between two elements of a dose-setting mechanism and a drive mechanism of the device changes its state, e.g. from engaged to released or vice versa. When the clutch, e.g. formed by sets of meshing teeth on the two elements, is engaged, the two elements may be rotationally locked to one another and when the clutch is disengaged or released, one of the elements may be permitted to rotate relative to the other one of the two elements. One of the elements may be a drive element or drive sleeve which engages a plunger rod of the dose-setting and drive mechanism. The drive sleeve may be designed to rotate relative to the housing element during dose setting and may be rotationally locked relative to the housing element during dose delivery. The engagement between drive sleeve and plunger rod may be a threaded engagement. Thus, as the drive sleeve cannot rotate during dose delivery, axial movement of the drive sleeve relative to the housing element will cause the plunger rod to rotate. This rotation may be converted into axial displacement of the plunger rod during the delivery operation by a threaded coupling between plunger rod and housing element.

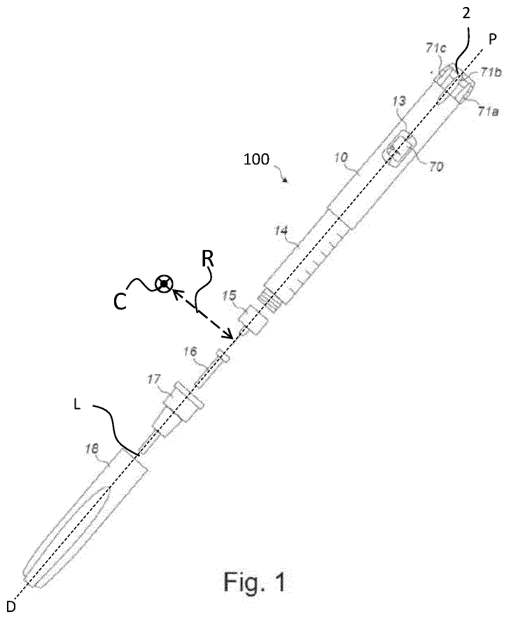

FIG. 1 is an exploded view of an exemplary embodiment of a drug delivery device 100. In this exemplary embodiment, the drug delivery device 100 is an injection device, e.g. a pen-type injector.

The injection device 100 of FIG. 1 is an injection pen that includes a housing element 10 holding a drug container 14, e.g. an insulin container, or a container holder for such a container 14. The container 14 may contain a drug, e.g. insulin. The container 14 may be a cartridge or a receptacle for a cartridge which may contain the cartridge or be configured to receive the cartridge. A needle 15 can be affixed to the container 14 or the receptacle. The container 14 may be a cartridge and the receptacle may be a cartridge holder. The needle 15 is protected by an inner needle cap 16 and either an outer needle cap 17 or another cap 18. An insulin dose to be ejected from the injection device 100 can be set, programmed, or ‘dialled in’ by turning a user interface member 2 in form of a knob 2, and a currently programmed or set dose is then displayed via dosage window 13, for instance in multiples of units. The units may be determined by the dose-setting mechanism which may permit relative rotation of the knob 2 to the housing element 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 100 is configured to administer human insulin, the dosage may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing element 10, which permits a user to view a limited portion of a dial sleeve 70 that is configured to move when the knob 2 is turned, to provide a visual indication of a currently programmed dose. The knob 2 is rotated on a helical path with respect to the housing element 10 when turned during programming.

In this exemplary embodiment, the knob 2 includes one or more features 71a, 71b, 71c in form of formations to facilitate gripping and/or attachment of a data collection device or electronic system.

The injection device 100 may be configured so that turning the knob 2 causes a mechanical click sound to provide acoustical feedback to a user. In this embodiment, the knob 2 also acts as an injection button. When needle 15 is stuck into a skin portion of a patient, and then the knob 2 is pushed in an axial direction, the insulin dose displayed in display window 13 will be ejected from injection device 100. When the needle 15 of injection device 100 remains for a certain time in the skin portion after the knob 2 is pushed home, the dose is injected into the patient's body. Ejection of the insulin dose may also cause a mechanical click sound, which is however different from the sounds produced when rotating the knob 2 during dialing of the dose.

In this exemplary embodiment, during delivery of the insulin dose, the knob 2 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

The injection device 100 may be used for several injection processes until either the insulin container 14 is empty or the expiration date of the medicament in the injection device 100 (e.g. 28 days after the first use) is reached.

Furthermore, before using injection device 100 for the first time, it may be necessary to perform a so-called “prime shot” to ensure fluid is flowing correctly from insulin container 14 and needle 15, for instance by selecting two units of insulin and pressing knob 2 while holding the injection device 100 with the needle 15 upwards. For simplicity of presentation, in the following, it will be assumed that the ejected amounts substantially correspond to the injected doses, so that, for instance the amount of medicament ejected from the injection device 100 is equal to the dose received by the user.

As explained above, the knob 2 also functions as an injection button so that the same component is used for dialling/setting the dose and dispensing/delivering the dose. Again, we note that a configuration with two different user interface members which, e.g., only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member can be moved relative to the body or housing of the device. During dose setting, the user interface member is moved proximally and/or rotates relative to the housing. During dose delivery, the user interface member moves axially, e.g. distally, e.g., without rotating relative to the housing or body.

FIG. 1 also indicates the coordinate system used herein for specifying positions of members or elements or features. The distal direction D and proximal direction P run parallel to the longitudinal axis L. The longitudinal axis L is a main extension axis of the device 100. The radial direction R is a direction perpendicular to the longitudinal axis L and intersecting with the longitudinal axis L. The azimuthal direction C, also referred to as angular direction or rotational direction, is a direction perpendicular to the radial direction R and to the longitudinal axis L. The different directions and axes will not be indicated in every of the following figures in order to increase the clarity of the figures.

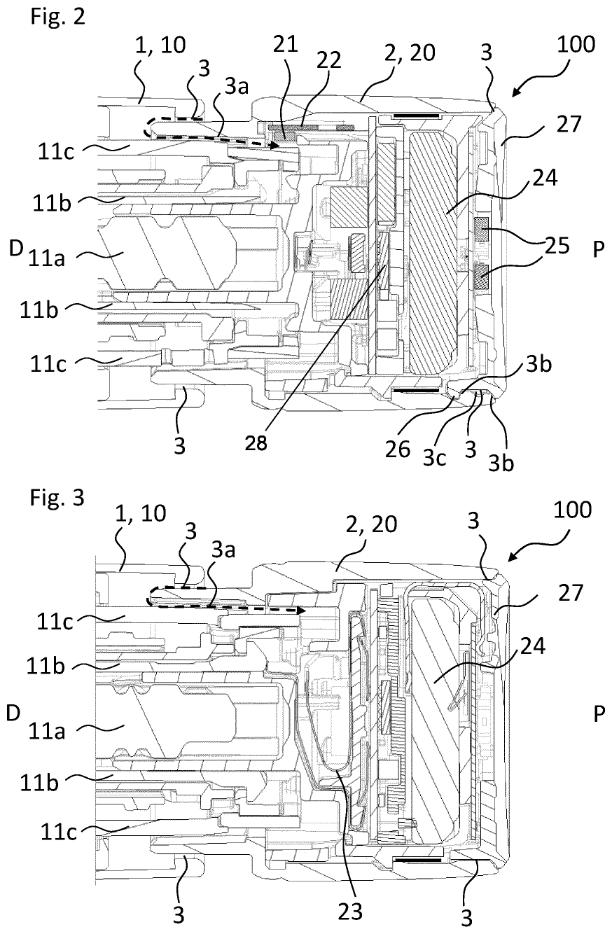

FIG. 2 shows a proximal section of the drug delivery device 100 of FIG. 1 in a cross-sectional view. FIG. 3 shows the same proximal section but in a different cross-sectional view. As can be seen, the component 2 in form of the knob 2 is connected to an arrangement 1, wherein the arrangement 1 includes the above-mentioned housing element 10. The knob 2 may be permanently or releasable connected to the arrangement 1.

The arrangement 1 includes, besides the housing element 10, a plunger rod 11a, a drive sleeve 11b and the dial sleeve 11c. These elements are operatively coupled for the dose dial and dose injection. During dose injection, the dial sleeve 11c rotates together with the number sleeve 70, for example. This rotation is detected by an optical sensor 21 in the interior of the knob 2 which does not rotate during dose injection.

The knob 2 includes, besides the optical sensor 21, further functional elements in its interior, namely a circuit board 22, an electro-mechanical switch 23, a battery 24, one or more LEDs 25 and/or a processor 28. The circuit board 22 may be electrically connected to the optical sensor 21 in order to receive the measurement signals of the sensor 21 and/or to provide the sensor 21 with electric power the circuit board 22 may be conductively connected to the processor. The battery 24 may be used to power the sensor 21, the LEDs 25 and/or further electric or electronic elements on the circuit board 22, e.g. the processor 28. The mechanical switch 23 may be operated by the drive sleeve 11b when the knob 2 is axially, e.g. distally, moved relative to the drive sleeve 11b. Operating the mechanical switch 23 may result in powering on the sensor 21 and/or the LEDs 25. The LEDs 25 may be configured to communicate to a user an operation state of the drug delivery device 100. For this purpose, a cover element 27 forming the proximal surface of the knob 2 may include a transparent region through which light of the LEDs 25 can leave the knob 2. The transparent region is, e.g., formed between the lateral surface of the knob 2 and the proximal surface of the knob 2.

The functional elements 21 to 25 in the interior of the knob 2 could be damaged or at least affected by fluid migrating from the exterior into interior of the knob 2, e.g., because these elements are associated with electric or electronic functionalities. Indeed, for the knob 2 shown in FIGS. 2 and 3, fluid could potentially reach into the interior and could reach to the functional elements 21 to 25 via transition regions 3 which are formed between facing surfaces of separate elements of the drug delivery device 100.

One such transition region 3 is formed between elements of the arrangement 1 and a housing element 20 in form of a grip element 20 of the knob 2. To be more precise, the transition region 3 is formed between a distal section of the grip element 20 projecting in distal direction D and a proximal section of the housing element 10 of the arrangement 1 projecting in proximal direction P as well as between the distal section of the grip element 20 and a proximal section of the dial sleeve 11c. Thus, the distal section of the grip element 20 is radially arranged between the housing element 10 and the dial sleeve 11c. The transition region 3 between the element 10, 20, 11c includes a gap. A fluid path 3a may extend through this gap. Fluid, e.g. water or drug, may therefore reach from the exterior into the interior of the knob 2 and can reach, e.g., the sensor 21. The fluid path 3a is indicated by the dashed line in FIGS. 2 and 3. A typical width or height of the gap may be 0.25 mm.

As can be seen in FIGS. 2 and 3, the fluid path 3a inside the transition region 3 includes two sections which extend in opposite axial directions. Fluid reaching from the exterior into the interior first has to travel in distal direction D along a first section of the fluid path 3a, then has to pass a coil or curved section of the fluid path 3a, respectively, and then has to travel in proximal direction P along a second section of the fluid path 3a, which may have a length of about 10 mm. In other words, the design of the transition region 3 is such that a tortuous fluid path 3a is created. This reduces the risk of fluid, e.g. liquid, indeed reaching from the exterior into the interior to any of the functional elements 21 to 25.

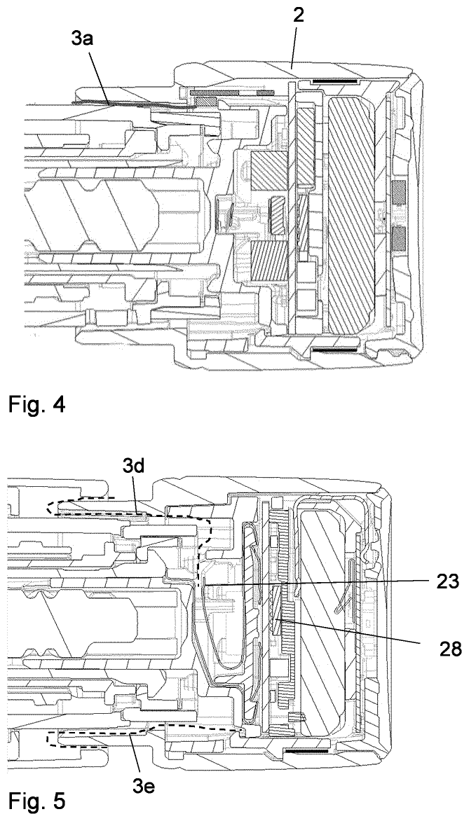

At this point it should be remembered that the knob 2 is axially and, e.g., rotationally movable with respect to the housing element 10 of the arrangement 1, e.g. to set a dose. To allow a good movability, a transition region 3 between the housing element 10 and the knob 2 can have a sufficiently large gap. However, this results in the fluid path 3a and a potential entry of fluid. Nevertheless, the special design of the transition region 3 reduces the risk of fluid reaching into the interior of the component 2, as will be explained in the following. When the knob is moved, e.g. for dose setting in the proximal direction away from the housing element 10, the length of the fluid path decreases, e.g. by the overlap between a skirt portion of the knob 2 (which may define the coiled or curved section of the fluid path 3a) separating the sections of fluid path extending in opposite directions and the housing element 10. When the dose has been set, the fluid path may be about 3 mm shorter than when no dose has been set as the knob has been moved away from the housing element axially. However, the no dose set or zero dose position of the knob 2 is the standard position of the knob and, hence, it is advantageous that the device is better protected against fluid travel into the relevant region(s) in the no dose or zero dose position of the knob given the longer fluid path. FIG. 4 illustrates fluid path 3a in the dose set position of the knob 2, when the knob has been moved proximally relative to the housing element 10 for a dose setting operation.

Further fluid paths, e.g. from the exterior of the device to the switch 23 (path 3d, e.g. about 13 mm) or towards a the processor 28 or a compartment within the knob wherein the processor 28 is arranged (path 3e, e.g. about 12 mm) are highlighted in FIG. 5. The processor 28 may be arranged on the same circuit board as the sensor 21 or on a separate circuit board. Fluid travelling along the path 3d may cause a malfunction, e.g. a short circuit, of the switch. Fluid travelling along the path 3e may impair the functionality of the processor or other elements or components in the compartment.

When axially moving the knob 2 with respect to the housing element 10, the surfaces of the elements 20, 10, 11c between which the transition region 3 is formed are also axially shifted with respect to each other. Thereby, also the length of the axially extending sections of the fluid path 3a change. For example, the section of the fluid path 3a extending in proximal direction P and lying more radially inwardly, changes its length.

In FIGS. 2 and 3, a further transition region 3 is formed at the proximal end of the knob 2, namely between the cover element 27 and the grip element 20 of the knob 2. These two elements 20, 27 are not movable with respect to each other. The transition region 3 between the two elements 20, 27 can therefore be formed tight, e.g. with a minimum or maximum distance between the surfaces of the two elements 20, 27 facing each other of, e.g., less than 0.05 mm. In this way, the entering of fluid from the exterior into the interior via this transition region 3 can be completely prevented. The length of this transition region may be about 2 mm, for example.

It can be seen in FIGS. 2 and 3 that the tight transition region 3 at the proximal end of the knob 2 includes one or more tight subregions 3b and, e.g. axially, arranged between the tight subregions 3b a non-tight subregion 3c, in which the distance/gap between the two elements 20, 27 is larger. A snap connection 26 between the elements 20, 27 adjoins the tight subregion 3b closer to the interior of the knob 2. The snap connection 26 mechanically connects and fixes the elements 20, 27 to each other by an interference fit.

As can be seen in FIGS. 2 and 3, some functional elements, e.g., the optical sensor 21 and the electro-mechanical switch 23, are arranged axially between the transition region 3 forming the fluid path 3a and the tight transition region 3 at the proximal end of the knob 2. For example, the functional elements are axially spaced from the transition regions 3 so that the probability that fluid reaches the functional elements is further reduced.

In the exemplary embodiment of FIGS. 2 and 3, surfaces of the elements defining the transition regions 3 and/or surfaces in the vicinity of the transition regions 3 may additionally or alternatively be formed by a hydrophobic material in order to further reduce the risk of fluid entering via this transition region 3. Hydrophobic materials may be fluoropolymer based materials and/or materials including or consisting of PTFE (polytetrafluoroethylene). The hydrophobic material may be or may include materials traded under Electrolube TCTF, 3M Novec 1700 or Acota Certonal FC-742.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The invention described herein is not limited by the description in conjunction with the exemplary embodiments. Rather, the invention includes any new feature as well as any combination of features, particularly including any combination of features in the patent claims, even if said feature or said combination per se is not explicitly stated in the patent claims or exemplary embodiments.

### REFERENCE NUMERALS

1 arrangement
2 component/user interface member/button/knob
3 transition region
3a fluid path
3b tight subregion
3c non-tight subregion
3d fluid path
3e fluid path
10 drug container holder/housing element
11a plunger rod
11b drive sleeve
11c dial sleeve
13 dosage window
14 drug container
15 needle
16 inner needle cap
17 outer needle cap
18 cap
20 grip element
21 optical sensor
22 circuit board
23 electro-mechanical switch
24 battery
25 LED
26 snap connection
27 cover element
28 processor
70 dial sleeve
71a . . . 71c formation
100 drug delivery device
D distal direction
P proximal direction
L longitudinal axis
R radial direction
C azimuthal direction/rotational direction/angular direction

1-18. (canceled)

19. A component for a drug delivery device, the component configured to be connected to an arrangement of the drug delivery device and comprising:
at least one functional element;
wherein at least when the component is connected to the arrangement, a transition region is formed between facing surfaces of elements of the drug delivery device, at least one of these elements being an element of the component;
wherein the transition region reaches from an exterior of the component to an interior of the component, and
wherein the transition region is designed such that a risk of fluid reaching from the exterior into the interior of the component via the transition region is reduced in order to protect the functional element.

20. The component according to claim 1, wherein the transition region is a fluid path transition region in which the facing surfaces are spaced from each other so that a fluid path along which fluid can travel extends between the facing surfaces, and wherein the transition region is designed such that, inside the transition region, the fluid path comprises two sections which extend in different axial directions.

21. The component according to claim 20, wherein inside the transition region, the fluid path comprises at least one coil and/or is a tortuous path.

22. The component according to claim 20, wherein the component is configured to be axially moved relative to at least one element of the arrangement during usage of the drug delivery device, and wherein the length of the fluid path inside the transition region is configured to change during movement of the component.

23. The component according to claim 19, wherein the transition region is formed between elements of both the component and the arrangement.

24. The component according to claim 23, wherein the transition region is formed between a distal section of the element of the component and a proximal section of an element of the arrangement, and wherein when the component is connected to the arrangement, the distal section of the element of the component and the proximal section of the element of the arrangement axially overlap and are offset from each other in radial direction.

25. The component according to claim 19, wherein the transition region is a fluid tight transition region in which the facing surfaces are tightly fitted to each other so that no fluid can pass through the transition region.

26. The component according to claim 19, wherein the transition region is formed between elements of the component.

27. The component according to claim 1, wherein the functional element is axially and/or radially offset from the transition region.

28. The component according to claim 19, wherein, at least when the component is connected to the arrangement, two transition regions are formed,
wherein one transition region of the two transition regions is a fluid path transition region and the other transition region of the two transition regions is a tight transition region, and
wherein the functional element is axially arranged between the fluid path transition region and the tight transition region.

29. The component according to claim 19, wherein a surface of the component in the vicinity of the transition region is formed of a hydrophobic material.

30. The component according to claim 19, wherein the functional element is associated with an electronic functionality of the component, and wherein the functional element is an optical sensor, electro-mechanical switch, circuit board, battery, or LED.

31. The component according to claim 19, wherein the component is a user interface member configured to be touched by a user in order to operate the user interface member when the component is connected to the arrangement.

33. The component according to claim 19, wherein the transition region reaches from an outer surface of the component to an inner surface of the component.

34. The component according to claim 33, wherein the inner surface of the component adjoins the interior of the component.

35. The component according to claim 19, wherein the transition region is a fluid path transition region in which the facing surfaces are spaced from each other so that a fluid path extends between the surfaces along which fluid can travel.

36. A drug delivery device comprising:
an arrangement comprising a container holder for holding a drug container; and
a component configured to be connected to the arrangement and comprising:
at least one functional element;
wherein at least when the component is connected to the arrangement, a transition region is formed between facing surfaces of elements of the drug delivery device, at least one of these elements being an element of the component;
wherein the transition region reaches from an exterior of the component to an interior of the component; and
wherein the transition region is designed such that a risk of fluid reaching from the exterior into the interior of the component via the transition region is reduced in order to protect the functional element.

37. The drug delivery device according to claim 36, wherein the component is a user interface member configured to be operated by a user in order to perform a dose dial and/or a dose delivery process, and wherein the component is configured to be rotated and/or axially moved relative to the container holder when operated by the user.

38. The drug delivery device according to claim 36, wherein the drug container comprises a drug.

39. A method of delivering a drug from a drug delivery device, the method comprising:
operating a drug delivery device, the drug delivery device comprising:
an arrangement comprising a container holder for holding a drug container; and
a component configured to be connected to the arrangement and comprising:
at least one functional element;
wherein at least when the component is connected to the arrangement, a transition region is formed between facing surfaces of elements of the drug delivery device, at least one of these elements being an element of the component;
wherein the transition region reaches from an exterior of the component to an interior of the component, and
wherein the transition region is designed such that a risk of fluid reaching from the exterior into the interior of the component via the transition region is reduced in order to protect the functional element.
